(19) United States
(12) Patent Application Publication
(10) Pub. No.: US 2026/0144664 A1
(43) Pub. Date: May 28, 2026

(54) ANIMAL SPINAL STABILIZATION SPLINT

(71) Applicant: Iowa State University Research Foundation, Inc., Ames, IA (US)

(72) Inventor: Rodney Steven Bagley, Stanhope, IA (US)

(21) Appl. No.: 19/395,445

(22) Filed: Nov. 20, 2025

Related U.S. Application Data
(60) Provisional application No. 63/725,711, filed on Nov. 27, 2024.

(51) Int. Cl. A61F 5/058

(52) U.S. Cl. CPC A61F 5/058

(57) ABSTRACT

A spinal stabilization splint is positioned on a four-legged animal. The splint securely and comfortably supports the spinal column of the animal to mitigate excessive spinal column manipulation or curvature of the spine, such as during patient movement, evaluation, and diagnostics. The splint can be used to address restraint for spinal injuries in generally any four-legged animal and can even include portions to allow for movement of the animal, such as a handle for lifting small animals while mitigating unwanted spinal column manipulation.

### CROSS REFERENCE TO RELATED APPLICATIONS

This application claims priority under 35 U.S.C. § 119 (e) to provisional patent application U.S. Ser. No. 63/725,711, filed Nov. 27, 2024. The provisional patent application is hereby incorporated by reference in its entirety herein, including without limitation: the specification, claims, and abstract, as well as any figures, tables, appendices, or drawings thereof.

### TECHNICAL FIELD

The present disclosure relates generally to apparatuses, systems, and/or methods of use having application in at least the veterinary and/or animal industry. More particularly, but not exclusively, the disclosure relates to apparatuses, systems, and/or methods for use in the care and recovery of animals, including dogs, having spinal injuries.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

When the spine of a four-legged animal, such as a dog, is injured, the most important concern is that spinal segments (articulating components of the spine) may be unstable—that is prone to excessive movement (see, e.g., FIGS. 1A-1C and 2A-2B). Excessive movement of the spinal column can result in increasing spinal cord (proper) damage. Therefore, in potentially spinal-injured patients, the spinal region requires external spinal support to prevent further injury during medical evaluation and care. Currently, there are few to no products that address this problem. In addition, when restraint is required to evaluate animals, it can be more likely that they arch or turn in a manner that can affect the injured spinal region.

Thus, there exists a need in the art for an apparatus that securely and comfortably support the spinal column of a four-legged animal.

### SUMMARY

The following objects, features, advantages, aspects, and/or embodiments are not exhaustive and do not limit the overall disclosure. No single embodiment needs to provide each and every object, feature, or advantage. Any of the objects, features, advantages, aspects, and/or embodiments disclosed herein can be integrated with one another, either in full or in part.

It is a primary object, feature, and/or advantage of any of the embodiments of the present disclosure to improve on or overcome the deficiencies in the art.

It is a further object, feature, and/or advantage of any of the embodiments of the present disclosure to provide spinal support for four-legged animals. For example, the support could be due to injury, evaluation, or other issues related to the animal's spine or spinal region.

It is still yet a further object, feature, and/or advantage of any of the embodiments of the present disclosure to minimize and mitigate excessive movement of the spinal column of a four-legged animal.

It is another object, feature, and/or advantage of any of the embodiments of the present disclosure to provide support to the spinal column/region of a four-legged animal.

It is yet another object, feature, and/or advantage of any of the embodiments of the present disclosure to provide a spinal support device that can be quickly and easily applied to a four-legged animal.

The apparatus disclosed herein can be used in a wide variety of applications. For example, the support/splint can be used during evaluation and/or care, as well as a way to move or restrain the animal while still providing support to the spinal column.

It is preferred the apparatus be safe, cost effective, and durable.

At least one embodiment disclosed herein comprises a distinct aesthetic appearance. Ornamental aspects included in such an embodiment can help capture a consumer's attention and/or identify a source of origin of a product being sold. Said ornamental aspects will not impede functionality of the apparatus.

According to some aspects of the present disclosure, an apparatus for providing spinal support and/or restraint to an animal comprises an elongated rigid member capable of corresponding to a spinal region of the animal; first and second wings extending from opposite sides of the elongated rigid member, the first and second wings rotatably connected to the elongated rigid member; and a first locking system associated with the first wing and a second locking system associated with the second wing, the first and second locking systems configured to hold the first and second wings in a closed manner at least partially surrounding the animal.

According to at least some embodiments, the first and second locking systems comprising loaded hinge mechanisms.

According to at least some embodiments, the loaded hinge mechanisms are adjustable to lock in position.

According to at least some embodiments, the apparatus further comprises a handle positioned on the elongated rigid member.

According to at least some embodiments, the elongated rigid member comprises multiple sections spaced from one another.

According to at least some embodiments, the apparatus further comprises first and second front shoulder supports extending from opposite sides of the elongated rigid member and configured to be positioned between a head and front legs of the animal.

According to at least some embodiments, the first and second locking systems are spring loaded.

According to at least some embodiments, the first and second locking systems comprise adjustable torque hinges.

According to at least some embodiments, the apparatus further comprises padding at the elongated rigid member and/or first and second wings.

According to at least some embodiments, the first and second wings are contoured in shape to the animal.

According to additional aspects of the disclosure, an animal spinal support comprises an elongated rigid member; a first wing extending laterally from the elongated rigid member, the first wing movable relative to the elongated rigid member; a second wing extending laterally from an opposite side of the elongated rigid member, the second wing movable related to the elongated rigid member; wherein the first and second wings extending from opposite sides of a central portion of the elongated rigid member; and wherein the first and second wings each including a locking member to lock the wings in place relative to the elongated rigid member.

According to at least some embodiments, the locking member comprises a loaded hinge mechanism.

According to at least some embodiments, the support further comprises a handle positioned on the elongated rigid member.

According to at least some embodiments, the support further comprises first and second front shoulder supports extending from opposite sides of the elongated rigid member at or near a front portion of the elongated rigid member.

According to at least some embodiments, the first and second front shoulder supports are movable relative to the elongated rigid member.

According to at least some embodiments, the first and second front shoulder supports comprise locking members to selectively lock the first and second shoulder supports in place.

According to at least some embodiments, the locking member comprises adjustable torque hinges.

According to at least some embodiments, the support further comprises padding at the elongated rigid member and/or first and second wings.

According to additional aspects of the disclosure, an animal support comprises an elongated rigid member; first and second wings extending from opposite sides of the elongated rigid member, the first and second wings movably connected to the elongated rigid member; and a first locking system associated with the first wing and a second locking system associated with the second wing, the first and second locking systems configured to hold the first and second wings in a selected configuration relative to the elongated rigid member; and first and second front shoulder supports extending from opposite sides of the elongated rigid member at a forward location of the first and second wings.

According to at least some embodiments, the first and second front shoulder supports comprise locking members to selectively lock the first and second shoulder supports in place.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIG. 1A is a close up depiction of a lateral radiographic image of a dog with a spinal injury resulting in spinal column instability.

FIG. 1B is another depiction of the radiographic image of the dog with spinal injury resulting in spinal column instability.

FIG. 1C is a depiction of a sagittal magnetic resonance image (MRI) similar to FIGS. 1A-1B, but also showing associated spinal cord damage resulting from spinal column instability.

FIG. 2A is a radiographic image of a dog with spinal injury showing undesired movement/displacement of the spinal column.

FIG. 2B is a close up image of FIG. 2A.

FIG. 3A is a depiction of an animal spinal column support apparatus according to aspects of the present disclosure.

FIG. 3B is a depiction of the apparatus of FIG. 3A on an animal.

FIG. 4A is a view of an animal with a spinal support apparatus in an open configuration according to aspects of the present disclosure.

FIG. 4B is a view similar to FIG. 4A, but with the spinal support apparatus in a closed configuration.

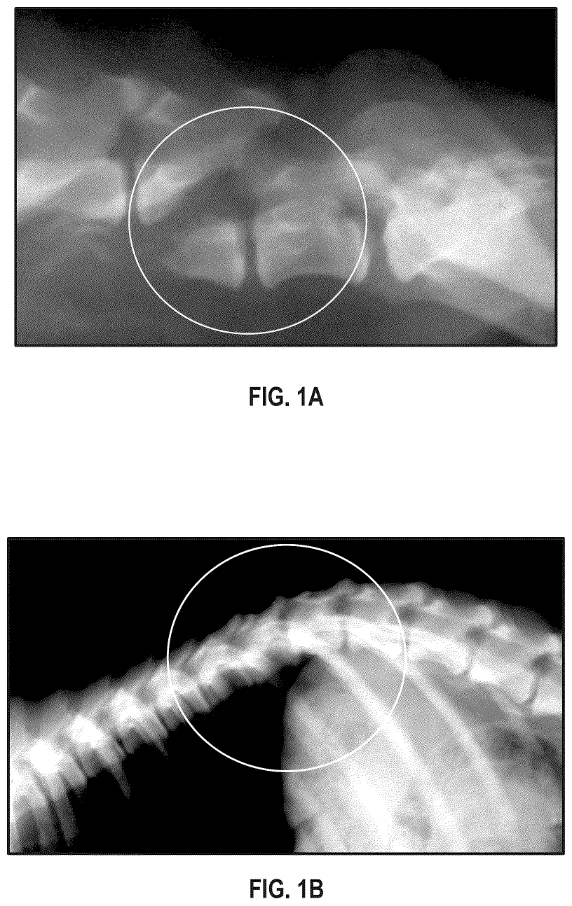

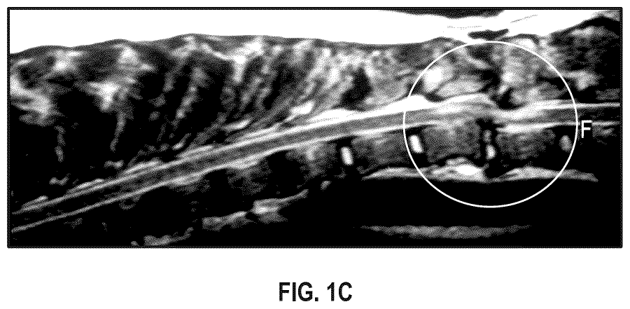

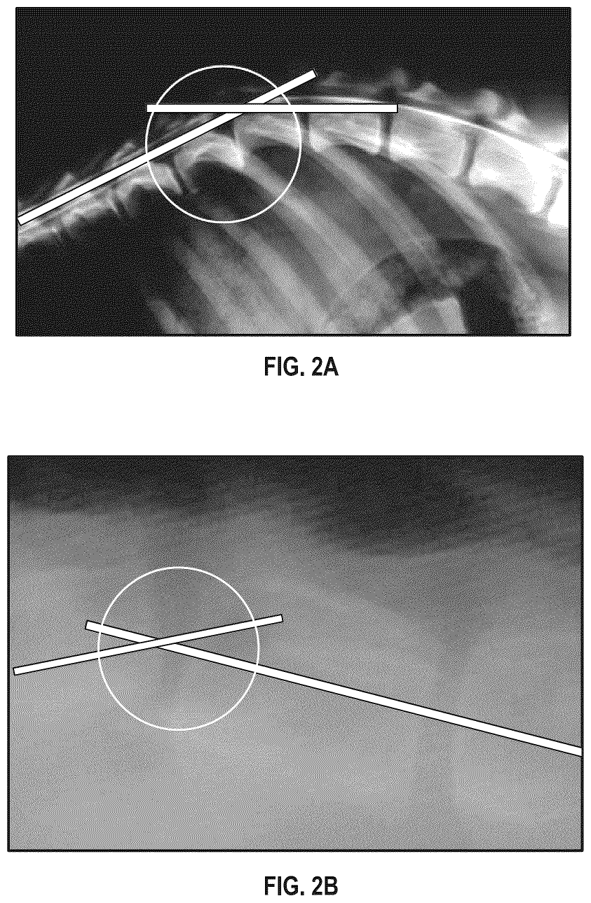

An artisan of ordinary skill in the art need not view, within isolated figure(s), the near infinite distinct combinations of features described in the following detailed description to facilitate an understanding of the present disclosure.

### DETAILED DESCRIPTION

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variables, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, sub-combinations, or the like that would be obvious to those skilled in the art.

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

As will be understood, the present disclosure includes aspects and/or embodiments for an animal spinal stabilization and restraint apparatus that securely and comfortably supports a four-legged animal's spinal column. According to at least some embodiments, the apparatus comprises a lightweight, rigid padded frame and a hinged opening and closing mechanism. As will be appreciated, at least some advantages of the apparatus is that once applied, the potential for excessive spinal column manipulation during patient movement, evaluation, and diagnostic is minimized, if not eliminated. Therefore, the apparatus can be utilized in acute spinal injury cases to align veterinary care protocols more closely with those used with humans. Equally, this device can be used for restraint. If restraint is required, the apparatus 10, by mitigating spinal bending, may also serve this purpose (i.e., restraint) in addition to spinal column immobilization.

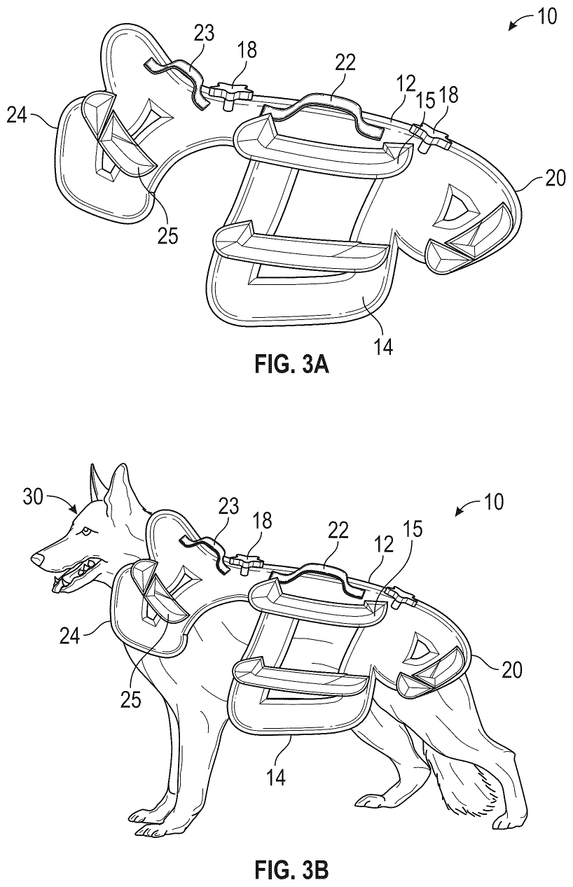

As shown in FIG. 3A, a spinal support apparatus 10 is shown. As will be understood, it is envisioned that the apparatus 10 could be varied in size to be used with generally any four-legged animal, and the size of the device would depend on the type of animal, breed of animal, age of animal, and size of the animal. For example, according to some embodiments, the device is used with dogs. However, this is not to be limiting on the disclosure and the use with any four-legged animal is to be considered.

According to some embodiments, the apparatus 10, such as shown in FIG. 3A, is a spinal stabilization splint. The apparatus 10 will include a rigid portion 12 for extending longitudinally along the spinal region of the animal 30. The rigid portion 12 can be a single component or multiple components connected to one another. For example, there can be parallel members along the spinal region of the animal. According to some embodiments, the rigid portion 12 can be a series of non-continuous components that are connected to one another such as to contact the spinal region of the animal at selected portions to stabilize the splint. In addition, there can be padding on the interior, such as at an interior side of the rigid portion 12 to aid in the comfort of the device for the animal.

Any rigid material can be used for the rigid portion 12. The apparatus may comprise any one or a combination of any known rigid materials, such as metals and metallic alloys, steel, plastics, composites, wood, stone, glass, and synthetic materials imitating the properties of any of the preceding materials.

According to some embodiments, a rigid plastic or polymer is used. In other embodiments, metals, such as hollow or non-hollow metal rods or tubes, could be used along the spinal region to provide the spinal support for the animal. Still further, plastic pipes, tubes, or rods could be combined and used to create the spinal support. Generally, any rigid material that can be formed or manipulated to extend along the spine of an animal should be considered to be used as part of the apparatus 10, such as at the rigid portion 12.

Extending from opposite sides of the elongated rigid portion 12 are first and second wing members 14 and 16. The wing members 14, 16 are connected to the rigid portion 12 in a movable manner, such as being rotatably connected to a portion of the elongated portion 12. The wing members 14, 16 can each comprise the same or a similarly rigid material as that of the elongated portion 12. The wings 14, 16 can also include padding or other material to aid in the comfort of the device when used with an animal.

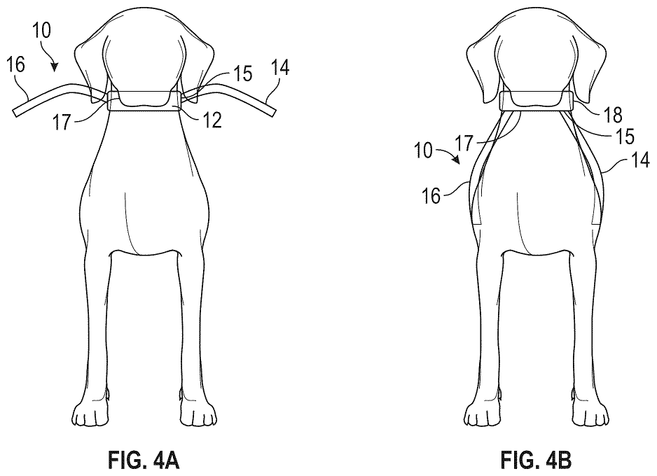

As noted, the wings 14, 16 are rotatably connected to the elongated rigid portion 12. This allows for a quick and easy positioning of the apparatus 10 on an animal. The movable wings allow the apparatus 10 to be articulated in open and closed positions. The open position (see, e.g., FIG. 4A) would allow for the apparatus to be placed from the dorsal perspective of the animal 30 and the closed configuration (see, e.g., FIG. 4B) ensures that the apparatus stays on the animal and provides the stabilization of the spinal region of the animal 30.

According to at least some embodiments, the wings 14, 16 are connected to the elongated portion 12 by hinges 15, 17, which may be lockable as well.

The hinged connection is typically established through a hinge comprising a pin, a knuckle, and a leaf. The pin is a rod that holds the leaves together inside the knuckle. The knuckle is a hollow and circular portion which creates a joint of the hinge through which the pin is set. The knuckles of either leaf typically alternate and interlock with the pin passing through all of them (i.e., a loop, joint, node, or curl). The leaf is the portion (there are typically two) that extends laterally from the knuckle and revolves around the pin.

The hinge can be characterized by its end play, gauge, hinge width, hinge length, knuckle length, leaf width, pitch, and slop. End play refers to the axial movement between the leaves along the axis of the pin. This motion allows the leaves to rotate without binding and is determined by the typical distance between knuckles (knuckle gap) when both edges of the leaves are aligned. Gauge refers to the thickness of the leaves. Hinge width refers to the length from the outer edge of one leaf to the outer ledge of the other leaf, perpendicularly across the pin (i.e., open width). Hinge length refers to the length of the leaves parallel to the pin. Knuckle length refers to the typical length of an individual knuckle parallel to the pin. Leaf width refers to length from the center of the pin to the outer edge of the leaf. Pitch refers to the distance from the end of a knuckle to the same edge of the adjacent knuckle on the same leaf. Slop refers to loose angular movement of the leaves relative to the pin.

Alternatively, the hinge may be a living hinge. A living hinge is a thin flexible hinge (flexure bearing) made from the same material as the two rigid pieces, e.g., [insert rigid pieces of the present invention], the living hinge connects. The living hinge is typically thinned or cut to allow the rigid pieces to bend along the line of the hinge. The minimal friction may make the living hinge more desirable in microelectromechanical systems or for connecting smaller rigid pieces.

More particularly, the hinge may comprise any known device which connects two solid objects, typically allowing only a limited angle of rotation between them. Some hinges aim to connect two objects such that rotation is relative about a fixed axis of rotation and all other translations or rotations are prevented (i.e., there is only one degree of freedom). The hinge or hinged connection may have varying amounts or levels of rotation, perhaps fixed by stoppers or other mechanisms which restrict movement.

The hinge may be made from substantially rigid materials such as steel, flexible materials such as flexure bearings, or even of moving components. Non-limiting exemplary types of hinges include spring hinges, barrel hinges, pivot hinges, butt/mortise hinges, case hinges, continuous hinges or piano hinges, concealed hinges, butterfly hinges, flag hinges, strap hinges, H hinges, HL hinges, and the like.

As noted, the hinges could include locking hinges that can be locked in place in either open or closed configurations. The locking of the hinges will aid in the function of the apparatus. For example, when locked in an open configuration, the apparatus 10 can be quickly and accurately positioned on the animal 30. Then, the wings 14, 16 can be closed at least partially around the animal and the wings can be locked in said closed configuration. This will keep the apparatus on the animal 30 to provide the support needed during movement, evaluation, diagnosis, and any other review of the animal.

To lock the wings 14, 16, the hinges 15, 17 may be spring-loaded hinges. Further, the hinges could be locking hinges or could include ratcheted to selectively lock the hinges. The locks could be button-activated, such as with quick release buttons to “unlock” any of the locking mechanisms to allow movement of the wings relative to the elongated portion. In addition, straps, ties, or other members could be used to connect the wings on the underside of the animal 30, which will provide additional securement of the apparatus on the animal. The locks may be adjustable, such as being shown by the adjustable members 18 in the figures. This can increase or decrease the tension of any springs of the hinges to control the movement of the wings relative to the spinal support portion 12.

Additional features shown in FIG. 3A and with the apparatus positioned on an animal 30 in FIG. 3B include multiple winged sections rotatably connected to the spinal support portion 12. As noted, the main wings 14, 16 extend at and at least partially around the midsection of the animal. There can be additional panels as well, which can include rotatably connected panels 24 at or near the neck region of the animal, and rotatably connected panels 20 at the rear section of the animal. These panels can be fixed in connected or movable, such as by rotation to the spinal support portion 12. When movable, the panels 20, 24 can include hinges 25, including locking hinges, to allow selective movement and control. In addition, straps can be included at the neck and/or rear portions to hold the apparatus on the animal 30. The additional panels can comprise the same material as the wings and can also include padding material on an interior portion to increase the comfort for the animal.

Still additional components include one or more handles 22, 23 along the rigid spinal portion 12. The handles can be affixed or otherwise connected to the apparatus to allow for controlled movement of the animal while the animal is “wearing” the support apparatus 10. The handles 22, 23 can also allow for lifting or other manipulation of the animal, while the apparatus maintains the spinal support to the animal 30.

FIGS. 4A and 4B show additional aspects of the spinal support apparatus 10. FIG. 4A shows the apparatus 10 in an open position, and FIG. 4B shows the apparatus 10 in a closed position. As noted, the loaded hinged mechanism would be adjustable to securely tighten externally around the spinal region. Once in place, the patient (i.e., animal) can be safely moved, turned, and manipulated for additional physical examination and diagnostic imaging.

Additional alternatives or options for the wings and panels are possible as well. As noted, ratchet members may be used with hinges or connections to selectively lock the panels in open/closed positions. Such locking and ratcheting hinges can include a quick release button. Buckles can be used to connect straps. Flexible, gripper arms can wrap around and contour with a portion of the animal's body. Such grippers can include articulating members that are essentially a series of connected, living hinges to provide more unlimited movement and contouring. These grippers can also be locked in place in open or closed positions, such as with screw/gear fasteners.

In addition, the wings or panels can take many forms. These can include frames made from plastic or other rigid portions, such as by connected PVC pipes to one another. Inflatable bladders can be used with the frames to better hold the apparatus in place and to provide comfort while wearing the apparatus. Tarps, cotton-stuffed materials, or generally any other material can be used as well to form the panels.

The following table may include some information for a suitable spinal support apparatus that will meet the objectives and goals for the apparatus. However, as will be understood, the following is included for example purposes and in no way should be considered to be limiting on the disclosure.

| Metrics | Goal | Units of Measure |
|---|---|---|
| Prototype patient capacity | 60-80 (Max 200) | LB |
| (weight capacity) | | |
| Materials - lightweight but | | |
| rigid (e.g., plastic) | | |
| Water/Chemical Resistant | | |
| (cleaners) | | |
| Opening and closing - | Angle of change | Degrees |
| mechanical with ratcheted | from 180° to 45° | |
| or motorized angulation | | |
| Force of closure/opening | Spring-loaded | |
| Patient comfort | Padded | |
| Health care provided | Handles | |
| comfort | | |
| Weight of apparatus | <25 | LB |
| Sound upon application | Silent | |

Therefore, a spinal support apparatus for animals has been shown and described herein. It should be appreciated that the disclosure should not be limited to the explicit figures shown, and that many variations, alternatives, and/or changes be made to any of the portions of any of the devices as shown and/or described. The device is not to be considered a single embodiment, and any of the aspects of any of the embodiments shown or described could be combined, even if not explicitly done herein, to create yet additional embodiments that provide the same or additional improvements and advantages for patients.

1. An apparatus for providing spinal support and restraint to an animal, comprising:
an elongated rigid member capable of corresponding to a spinal region of the animal;
first and second wings extending from opposite sides of the elongated rigid member, the first and second wings rotatably connected to the elongated rigid member; and
a first locking system associated with the first wing and a second locking system associated with the second wing, the first and second locking systems configured to hold the first and second wings in a closed manner at least partially surrounding the animal.

2. The apparatus of claim 1, wherein the first and second locking systems comprising loaded hinge mechanisms.

3. The apparatus of claim 2, wherein the loaded hinge mechanisms are adjustable to lock in position.

4. The apparatus of claim 1, further comprising a handle positioned on the elongated rigid member.

5. The apparatus of claim 1, wherein the elongated rigid member comprises multiple sections spaced from one another.

6. The apparatus of claim 1, further comprising first and second front shoulder supports extending from opposite sides of the elongated rigid member and configured to be positioned between a head and front legs of the animal.

7. The apparatus of claim 1, wherein the first and second locking systems are spring loaded.

8. The apparatus of claim 1, wherein the first and second locking systems comprise adjustable torque hinges.

9. The apparatus of claim 1, further comprising padding at the elongated rigid member and/or first and second wings.

10. The apparatus of claim 1, wherein the first and second wings are contoured in shape to the animal.

11. An animal spinal support, comprising:
an elongated rigid member;
a first wing extending laterally from the elongated rigid member, the first wing movable relative to the elongated rigid member;
a second wing extending laterally from an opposite side of the elongated rigid member, the second wing movable related to the elongated rigid member;
wherein the first and second wings extending from opposite sides of a central portion of the elongated rigid member; and
wherein the first and second wings each including a locking member to lock the wings in place relative to the elongated rigid member.

12. The animal spinal support of claim 11, wherein the locking member comprises a loaded hinge mechanism.

13. The animal spinal support of claim 11, further comprising a handle positioned on the elongated rigid member.

14. The animal spinal support of claim 11, further comprising first and second front shoulder supports extending from opposite sides of the elongated rigid member at or near a front portion of the elongated rigid member.

15. The animal spinal support of claim 14, wherein the first and second front shoulder supports are movable relative to the elongated rigid member.

16. The animal spinal support of claim 15, wherein the first and second front shoulder supports comprise locking members to selectively lock the first and second shoulder supports in place.

17. The animal spinal support of claim 11, wherein the locking member comprises adjustable torque hinges.

18. The animal spinal support of claim 11, further comprising padding at the elongated rigid member and/or first and second wings.

19. An animal support, comprising:
an elongated rigid member;
first and second wings extending from opposite sides of the elongated rigid member, the first and second wings movably connected to the elongated rigid member; and
a first locking system associated with the first wing and a second locking system associated with the second wing, the first and second locking systems configured to hold the first and second wings in a selected configuration relative to the elongated rigid member; and
first and second front shoulder supports extending from opposite sides of the elongated rigid member at a forward location of the first and second wings.

20. The animal support of claim 19, wherein the first and second front shoulder supports comprise locking members to selectively lock the first and second shoulder supports in place.
